(19) United States
(12) Patent Application Publication
(10) Pub. No.: US 2008/0183103 A1
(43) Pub. Date: Jul. 31, 2008

(54) BULBOUS DISTAL ENDED CATHETER

(75) Inventors: Drew Blankenship, Lake Elsinore, CA (US); Eric T. Johnson, Temecula, CA (US)

Correspondence Address: FAEGRE & BENSON, LLP;32469, 2200 WELLS FARGO CENTER, 90 SOUTH SEVENTH STREET, MINNEAPOLIS, MN, 55402-3901, US

(73) Assignee: Cardiac Pacemakers, Inc., St. Paul, MN (US)

(21) Appl. No.: 11/626,948

(22) Filed: Jan. 25, 2007

(51) Int. Cl. A61M 25/01

(52) U.S. Cl. 600/585

(57) ABSTRACT

The present invention is a shaped catheter having an atraumatic tip portion which facilitates ease of navigation through the patient's vascular system. The catheter includes a shape-region formed at or near the distal end of the catheter. The shape is formed by placing the catheter over a shaping tool that includes a die, heating the catheter to its softening point for a desired period of time, and then cooling the catheter. A shrink tubing may also be placed over the catheter after it is placed on the shaping tool and before heating to assist in achieving the desired shape.

### TECHNICAL FIELD

The present invention relates to catheters for medical applications. More specifically, the present invention relates to a shaped catheter that with an atraumatic tip portion which facilitates ease of navigation through the patient's vascular system, and methods for making such a catheter.

### BACKGROUND

Catheters are commonly introduced into veins and routed through the venous system to selected locations. Materials may be introduced through the catheter or injected into the body as part of a diagnostic or treatment procedure. Typically, such catheters have an elongated, flexible body of a predetermined diameter smaller than the cross-sectional diameter of the vessels through which the catheter must pass. Catheters may, for example, be used to deliver medical electrical leads (e.g., for use in performing cardiac rhythm management). These leads may include a flexible sheath surrounding elongated conductors that terminate at a distal end in one or more surface electrodes adapted to contact heart tissue. Catheters may also be utilized for other procedures in the body, such as, for example, urinary catheterizations, angioplasty, and sinoplasty.

Catheters may be formed from various biocompatible materials including, without limitation, polyethylene, polyurethane, polypropylene, and silicone rubber. The catheters must have sufficient structural integrity such that they can pass through the vascular system to the desired location. Some catheters may be harder or stiffer at the proximal end and other catheters may be softer or more flexible at the distal end. Other catheters may be of a uniform stiffness or durometer throughout the length. Catheters are desirably configured to have atraumatic distal ends to avoid trauma to the patient's vascular tissue. In addition, catheters are desirably configured so as to readily navigate the various turns and branches in the patient's vascular system.

A need therefore exists for improved catheters with atraumatic distal ends and for catheters configured to facilitate enhance ease of navigation through the patient's vascular system.

### SUMMARY

The present invention, according to one embodiment, is a method of forming a shaped catheter. The method includes selecting an elongated flexible tubular catheter including a lumen extending through a length thereof, selecting a shaping tool, the shaping tool including a shaft with an outside diameter approximately equal to an inside diameter of the lumen of the tubular catheter, the shaft further including a die at a selected position and being of a selected dimension, inserting the shaping tool into the tubular catheter a desired distance, covering the catheter with a shrink tube, heating the shrink tubing and at least that portion of the tubular catheter surrounding the die to a desired temperature, cooling the tubular catheter such that the catheter takes on the shape of the shaping tool, and removing the shrink tubing and the shaping tool.

The present invention, according to another embodiment, is a kit for forming a shaped elongated tubular member with a lumen, the elongated tubular member including an inner diameter and an outer diameter, a shaping tool including a shaft with an outside diameter approximately equal to the inside diameter of the lumen of the elongated tubular member, the shaping tool including a die of a desired shape positioned on the shaft, and a shrink tube, the shrink tube including a lumen with a first inner diameter greater than the outer diameter of the elongated tubular member, the shrink tube adapted to shrink upon heating whereby the shrink tube after shrinking includes a second inner diameter of a desired width.

The present invention, according to yet another embodiment, is a shaping tool for molding an elongated tubular member into a desired shape. The shaping tool includes a cylindrical member with a first portion of an outside diameter approximately equal to an inner diameter of a lumen of the elongated tubular member and a second portion of an outside diameter greater than the inner diameter of the lumen of the elongated tubular member and a die of a selected contour disposed at a position on the first portion of the cylindrical member proximal to the second portion of the cylindrical member.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a side elevational cut away view of one embodiment of the present invention placed on a shaping tool.

FIG. 2 is a side view of a distal portion of the catheter of FIG. 1.

FIG. 3 is cross-sectional elevation view of a portion of the catheter of FIG. 1.

FIG. 4 is a schematic illustration of the distal portion of the catheter of FIG. 1 navigating an exemplary region of the patient's vasculature.

FIG. 5 is a side view of a portion of an alternative shaped catheter according to another embodiment of the present invention.

FIG. 6 is a side view of a portion of an alternative shaped catheter according to another embodiment of the present invention.

FIGS. 7 and 8 are side and cross-sectional views, respectively, illustrating a method of forming a shape-region of the catheter of FIG. 1 using a shaping tool according to one embodiment of the present invention.

FIG. 9 is a perspective view of a portion of an alternative shaping tool for forming a shape-region of a catheter according to another embodiment of the present invention.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

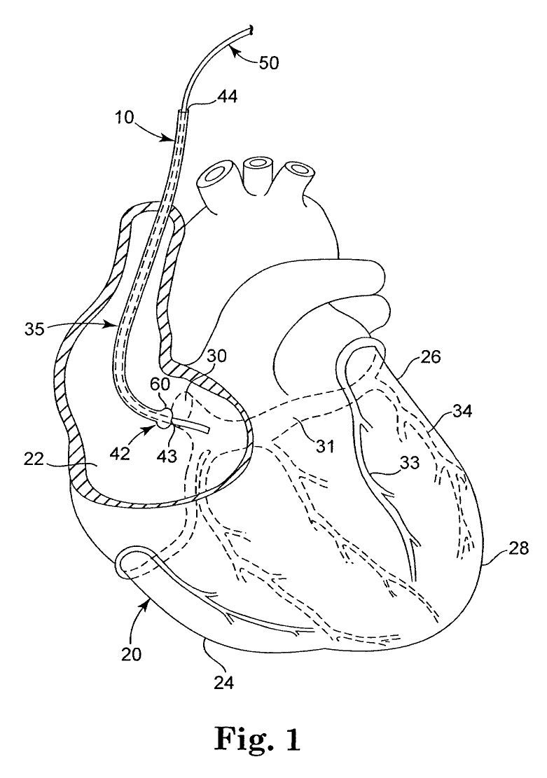

FIG. 1 is a schematic drawing illustrating a catheter 10 according to one embodiment of the present invention positioned partially in a patient's heart 20. As is known, the heart 20 includes a right atrium 22 and a right ventricle 24, a left atrium 26 and a left ventricle 28, a coronary sinus ostium 30 in the right atrium 22, a coronary sinus 31, and various coronary veins including a great cardiac vein 33 and other branch vessels off the coronary sinus 31 including an exemplary branch vessel 34. As shown in FIG. 1, the catheter 10 includes an elongate proximal catheter body 35, a distal tip portion 42 extending distally from the body 35 and terminating in an open distal end 43, and a lumen 44 extending through the body 35 and the distal tip portion 42 to the distal end 43. In the embodiment illustrated in FIG. 1, the catheter 10 is guided through the patient's vasculature and the right atrium 22 and into the coronary sinus 31 through the coronary sinus ostium 30, and an inner guide catheter 50 is disposed within the lumen 44 and extends distally beyond the distal end 43 for cannulating the coronary sinus 31 and branch vessels thereof. The catheter 10 may, in other embodiments, be used for other catheterization procedures or to deliver other payloads including, without limitation, medical electrical leads, guide wires, introducers, drugs, contrast media and the like. Additionally, in other embodiments, the catheter 10 can be used in other regions of the patient's vascular system or in other body lumens. As shown in FIG. 1, the distal tip portion 42 includes at least one shaped region 60 proximate the distal end 43. As will be shown and explained in greater detail below, the shaped region 60 operates as an atraumatic distal end feature and enhances ease of placement and the vessel access capabilities of the catheter 10.

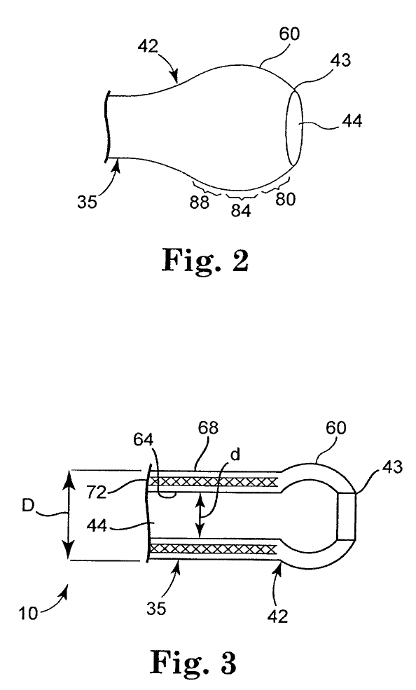

FIGS. 2 and 3 are side and cross-sectional views, respectively, of the distal tip portion 42 and the distal-most portion of the body 35 of the catheter 10. As shown, the catheter 10 has an inner surface 64 defining a diameter d of the lumen 44, and an outer surface 68 defining an outer diameter D of the catheter 10. As will be appreciated, the catheter diameter D is selected to be smaller than the diameter of the blood vessels (or other bodily lumens) through which the catheter will be advanced. As further illustrated, both the lumen diameter d and the catheter diameter D in the shaped region 60 are generally larger than in the catheter body 35 in general. In the illustrated embodiment, the body 35 includes a metallic, e.g., stainless steel, braid 72 embedded between the inner and outer surfaces 64, 68 and terminating at a location proximal to the shaped region 60. When present, the metallic braid 72 operates to stiffen and reinforce the portions of the catheter 10 in which it is located. In various embodiments, the braid 72 may further extend partially into the distal tip portion 42. In still other embodiments, the braid 72 may be omitted.

As shown in FIG. 2, the shape-region 60 includes, in the illustrated embodiment, a first curve 80, a second curve 84, and a third curve 88. The first curve 80 defines the contour or profile of the distal (leading) portion of the shape-region 60 relative to the body 35 and the catheter 10 in general. The second curve 84 is generally the middle portion of the shape-region 60, and the third curve 88 is the proximal portion of the shape-region 60 that re-joins the body 35 of the catheter 10. In the embodiment shown, the first, second, and third curves 80, 84, 88 present a generally uniform (i.e., spherical) profile such that the inner surface 64 in the shape-region 60 forms a substantially uniform, spherical concave surface. In alternative embodiments, the first curve 80, second curve 84, and/or third curve 88 may present generally shallow, flat, or steeply inclined angles that vary from one section to the next or that are all generally similar.

The catheter 10 can be dimensioned for use in any catheterization procedure. In various embodiments, the catheter 10 may be a guide catheter for delivering pacing and/or defibrillation leads for applying electrical stimuli to one or more chambers of the heart. For example, in the embodiment illustrated in FIG. 1, the catheter is an outer guide catheter configured for accessing the coronary sinus for delivering pacing leads for stimulating the left side of the heart. In one embodiment, the catheter diameter D ranges from 4 French to 13 French. In one embodiment, the catheter 10 is an 8 French catheter having an outer diameter D of about 0.105 inches and a lumen diameter d of about 0.087 inches, and includes a soft tip that is about 4 mm in length. Other catheters 10 of different diameters, thicknesses, and tip lengths may be likewise utilized. The shape-region 60 may be further incorporated into other medical devices such as guidewires, sheaths, stents, catheters, delivery devices, tubes, other elongated tubular members, and introducers.

The shape-region 60 may take any desired dimensions. The shape-region 60, as further discussed below, may range from a slight increase to a fairly distinct and/or significant increase in the diameter D of the catheter 10. In one embodiment, the total length of the shape from the beginning of the first curve 80 through the end of the third curve 88 may be from about 0.10 to about 0.20 inches. Of course, in other embodiments, the shape-region 60 may have a longer or shorter length. That is, in various embodiments, the length and/or the largest outer diameter of the shape-region 60 may be varied as desired. In other embodiments, the shape-region 60 may include a generally consistent curve around the circumference of the shape-region 60. In still further embodiments, the shape-region 60 may include different forms or curves that are presented on different sides of the catheter 10. Further embodiments of the shape-region 60 may have flat, conical, or pear-shaped profiles.

In various embodiments, the portion of the catheter 10 at the distal end 43 of the shape-region 60 may include a portion that is narrower than lumen diameter d of the catheter body 35. The narrower open end may act as a seal between the catheter 10 and the inner catheter 50 (see FIG. 1) or other payload extending there through.

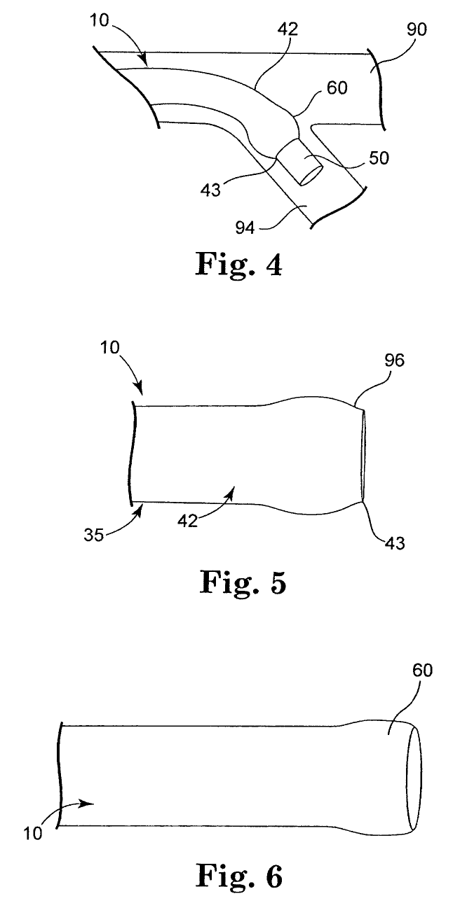

FIG. 4 is a schematic illustration of the distal tip portion 42 of the catheter 10 navigating an exemplary region of the patient's vasculature including a main vessel 90 and a branch vessel 94. As can be seen in FIG. 4, the shape-region 60 presents an arcuate, contoured leading surface of the distal tip portion 42, and also operates to separate the distal end 43 from the surfaces of the vessels 90 and 94 during placement. Additionally, because of the arcuate surface presented on the leading portion of the shape-region 60, less force is required to collapse the shape-region 60 in on itself as compared to relatively blunt-ended catheters of conventional design. The foregoing features advantageously help to keep the distal end 43 of the catheter 10 from catching on the interior anatomy as it passes through. As further illustrated in FIG. 4, the contoured leading profile of the shape-region 60 may further allow the catheter 10 to more easily track through splits, junctures, and curves in the vascular system. Moreover, because the shape-region 60 operates to separate the distal end 43 from the vessel tissue, the inner guide catheter 50 or other payload extending through the distal end 43 is also less prone to engaging or catching on the vessel tissue as it is advanced distally.

In some embodiments, the shape-region 60 may facilitate improved fluoroscopy procedures by improving the flow of contrast media in certain areas of the anatomy by creating a seal between the anatomy wall and the outer surface of the shape-region 60. In various embodiments, the particular dimensions of the shape-region 60 may be designed to collapse at a certain threshold pressure such that it can be inserted as far into smaller portions of the vascular anatomy as a catheter without such a shape-region 60.

The shape-region 60 may be located at any position along the length of the catheter 10. FIGS. 5 and 6 illustrate alternative embodiments of contours and placement positions of the shape-region 60. In an alternative embodiment of the catheter 10 shown in FIG. 5, the distal tip portion 42 includes a portion 96 between the shape-region 60 and the distal end 43. In one embodiment, the portion 96 has a diameter that is slightly narrower then the main body 35 of the catheter so as to present a reduced profile as the catheter 10 is advanced through the patient's vasculature. In the alternative embodiment shown in FIG. 6, the shape-region 60 is located at the very distal end of the catheter 10 so as to form a generally bell shaped distal end. As will be appreciated, the shape-region 60 can have other profiles and can be located at other positions along the catheter 10 and dictated by the particular clinical use for the catheter 10

The catheter 10, including the catheter body 35, may be made from any materials known in the art or later developed for medical catheters. Exemplary materials include, without limitation, polyethylene, polyurethane, polypropylene, silicone rubber, and polyether block amides sold under the brand name Pebax® In various embodiments, the body 35 and the distal tip portion 42 may be made from the same material, may have a substantially monolithic structure, or may be made of different materials and/or separate components joined together using any methods known in the art, e.g., adhesive bonding. In the illustrated embodiment, the body 35 and the distal tip portion 42, including the shape-region 60, are made from the same material. In one embodiment, the body 35 and the distal tip portion 42 may be of the same or substantially the same durometer or may have different durometers. In one embodiment, the distal tip portion 42 may be relatively soft and flexible as compared to the body 35 in general. In one embodiment, the body 35 may have regions of varying durometer along its length. In one embodiment, the body 35 may be made of a lower durometer material near the distal tip portion 42 and the shape-region 60, and a relatively higher durometer material in its proximal region.

The catheter 10 may be manufactured by any suitable process. In one embodiment, the catheter 10 may be first formed of a selected plastic material through processes well known to those in the art. In one method of catheter 10, formation of a selected plastic may be melted and extruded over a stainless steel braided core 72. The plastic may then be shrunk, if necessary, by heating and through the application of a shrink jacket. The end may also be manicured as desired. The resultant catheter 10 may include the stainless steel braid 72 through a portion of the wall of the catheter 10. In some embodiments, the stainless steel braid 72 may be non-existent or substantially absent from that portion of the catheter 10 in which the shape-region 60 will be formed.

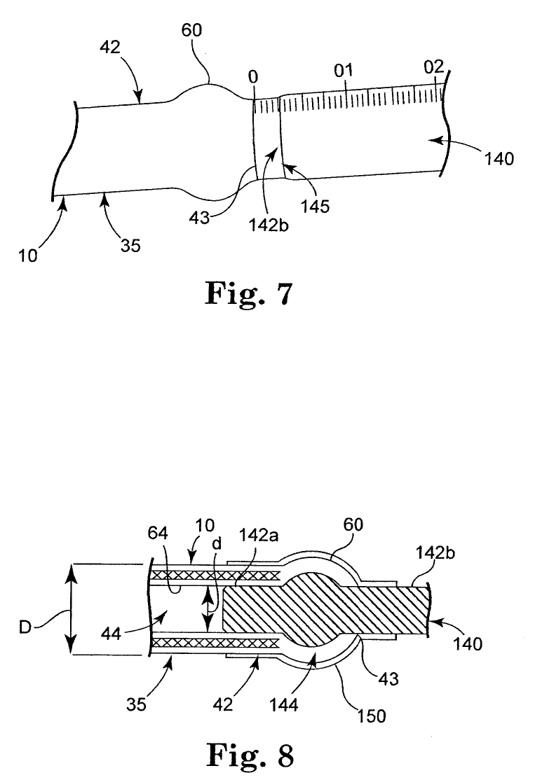

FIGS. 7 and 8 are side and cross-sectional views, respectively, illustrating a method of forming the shape-region 60 of the catheter 10 using a shaping tool 140 according to one embodiment of the present invention. As shown in FIGS. 7 and 8, the shaping tool 140 includes first and second shaft portions 142a, 142b, and a die 144 on the first shaft portion 142a proximal to the second shaft portion 142b. As can be seen in FIGS. 7 and 8, the shaping tool 140 is configured to be partially inserted into the catheter lumen 44 so as to form the shape-region 60. As such, the die 144 has a selected shape corresponding to the desired shape of the shape-region 60 to be formed into the distal tip portion 42 of the catheter 10, and the shaping tool 140 is configured such that the die 144 can be placed in the catheter 10 at the desired location of the shape-region 60. In the illustrated embodiment, the die 144 has a generally spherical shape so as to produce a generally spherical shape-region 60. It is emphasized, however, that the die 144 may take on any shape depending on the desired shape of the shape-region 60 to be formed in the catheter 10.

In the illustrated embodiment of FIG. 7, the shaft portion 142b has an increased-diameter portion forming an optional stop 145 which operates to promote consistent placement of the shape-region 60 at the desired location on the catheter 10. As further shown, the shaping tool 140 also includes optional measurement markings or other markings to indicate the extent of insertion of the shaping tool 140 into the catheter lumen 44 (see FIG. 8) during formation of the shape-region 60. In further embodiments, such measurement markings may be used in lieu of the stop feature 145. In other embodiments, neither the stop 145 nor the measurement markings may be present. In other embodiments, the shaping tool 140 may include other features to facilitate accurate and consistent placement of the shape-region 60.

In one embodiment, at least the shaft portion 142a may have a diameter substantially equal to or slightly smaller than the lumen diameter d. In one embodiment, the shaft portion 142b may have a smaller diameter than the lumen diameter d. The narrower diameter of the shaft portion 142b may advantageously cause the distal end 43 of the distal tip portion 42 to have a narrower open end than the lumen diameter d of the catheter body 35. In such an embodiment, the open distal end 43 can operate to sealably and slidably engage the outer surface of a payload, e.g., the inner catheter 50, as it is advance distally beyond the catheter distal end 43 (see FIG. 1). Such engagement may, in some embodiments, impede ingress of blood or other bodily fluids into the catheter lumen 44. This in turn may further help to reduce blood clotting and prevent the payload from sticking to the catheter inner surface 64 during delivery procedures.

The shaping tool 140 may be made out of polytetrafluorethylene (PTFE), acetal, polished stainless steel or other similar materials. Such materials may be useful in reducing contaminants or particles transferred to the catheter 10 and may also allow for easy removal of the shaping tool 140 once the formation of the shape-region 60 is completed.

As illustrated in FIGS. 7 and 8, during formation of the shape-region 60, the distal distal tip portion 42 of the catheter 10 may be placed over the shaping tool 140 to the stop 145 or to a desired distance from the stop 145. Next, a removable shrink tube 150 may be placed on the outer surface 68 of the catheter body 35 and second shaft portion 142b of the shaping tool 140. The shrink tube 150 may be a thermofit tubing such as a thermally stabilized polyolefin that is cross linked. The shrink tubing 150 may have a first inner diameter that is greater than the outer diameter D of the catheter 10 and a second diameter that approximately equal to or slightly smaller than the outer diameter D of the catheter 10. As illustrated in FIG. 8, the shrink tubing 150 is placed over the distal dip portion 42 of the catheter 10 and at least to the stop 145. Placing the shrink tubing 150 past the distal end 43 of the catheter 10 forms a seal between the shrink tubing 150 and the second shaft portion 142b of the shaping tool 140 to prevent portions of the softened catheter body material from running upon being heated. The shrink tubing 150 further facilitates effective control of the shape and thickness of the distal tip portion 142 of the catheter 10 at the distal-most portion of the shape-region 60.

The catheter 10 is then heated to soften the catheter body 35 and distal tip portion 42, shrink the shrink tubing 150, and form the shape-region 60. During heating, at least the portion of the distal tip portion 42 proximate the die 144 is heated to or above its softening temperature for a time sufficient to reform the distal tip portion 42. It will be appreciated that the temperature and holding time for this heating step will be determined primarily by the particular materials used for the distal tip portion 42 (or in other embodiments, the other portion of the catheter 10 in which the shape-region 60 is located).

In one embodiment, the distal tip portion 42 may be heated to approximately 300° F. for approximately 20 seconds. Such heating causes the shrink tubing 150 to shrink and squeeze the distal tip portion 42 so as to ensure that the distal tip portion 42 uniformly conforms to the shaping tool 140. The shrink tubing 150 may also help to insure a consistent thickness of the shape-region 60. After the catheter 10 is cooled, the shrink tubing 150 may be removed and the shaping tool 140 may be removed from the catheter lumen 44, leaving the shape-region 60 at the desired location. As may be appreciated, several of these steps are not dependent upon an order of completion.

In further embodiments where the catheter 10 is advanced over the shaping tool 140 to the stop 145 of a wider diameter than shaft 142a of shaping tool 140, the shrink tubing 150 may also be placed all the way to the stop 145. As may be appreciated, a number of combinations of placing the catheter 10 on the shaping tool 140 with the shrink tubing 150 may be realized.

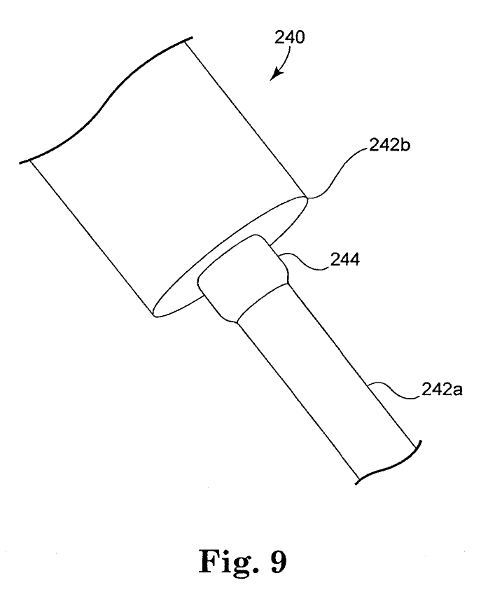

FIG. 9 is a perspective view of another exemplary shaping tool 240 according to another embodiment of the present invention. As shown in FIG. 9, the shaping tool 240 includes a proximal shaft portion 242a and an enlarged-diameter distal portion 242b operating as a stop similar to the stop 145 of the shaping tool 140. As further shown, the proximal shaft portion 242a includes a die 244 for forming the catheter shape-region. The embodiment illustrated in FIG. 9 includes a generally barrel-shaped die 244 with a generally straight side. As explained above, the die 244 can have any shape so to produce the desired shape of the catheter shape-region.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope he claims, together with all equivalents thereof.

We claim:

1. A method of forming a shaped catheter comprising:
selecting an elongated flexible tubular catheter including a lumen extending through a length thereof;
selecting a shaping tool, the shaping tool including a shaft with an outside diameter approximately equal to an inside diameter of the lumen of the tubular catheter, the shaft further including a die at a selected position and being of a selected dimension;
inserting the shaping tool into the tubular catheter a desired distance;
covering the catheter with a shrink tube;
heating at least that portion of the shrink tubing and the tubular catheter surrounding the die to a desired temperature;
cooling the tubular catheter such that the catheter takes on the shape of the shaping tool; and
removing the shrink tubing and the shaping tool.

2. The method of claim 1 wherein inserting the shaping tool further comprises inserting a shaping tool with a die in the shape of a ball.

3. The method of claim 1 wherein inserting the shaping tool further comprises inserting a shaping tool with a stop formed on a portion of the shaft.

4. The method of claim 3 wherein inserting the shaping tool further comprises inserting a shaping tool made from one or more of polytetrafluorethylene, acetal, or stainless steel.

5. The method of claim 1 wherein inserting the shaping tool further comprises inserting a shaping tool made of one or more of polytetrafluorethylene, acetal, or stainless steel.

6. The method of claim 1 wherein inserting the shaping tool further comprises inserting a shaping tool wherein the shaft includes a portion with a narrower diameter between the die and the stop, the narrow portion of a smaller diameter than the inside diameter of the lumen of the tubular catheter.

7. The method of claim 1 wherein heating the tubular catheter includes heating the tubular catheter to its softening point.

8. The method of claim 1 wherein selecting the elongated flexible tubular catheter includes selecting a catheter including a metallic braid in at least a portion thereof.

9. The method of claim 1 wherein selecting the elongated flexible tubular catheter includes selecting a catheter including a soft distal tip portion.

10. The method of claim 1 wherein covering the catheter with a shrink tube further comprises covering the catheter with a shrink tube of a pre-selected diameter.

11. A kit for forming a shaped elongated tubular member comprising:
a elongated tubular member with a lumen, the elongated tubular member including an inner diameter and an outer diameter;
a shaping tool including a shaft with an outside diameter approximately equal to the inside diameter of the lumen of the elongated tubular member, the shaping tool including a die of a desired shape positioned on the shaft; and
a shrink tube, the shrink tube including a lumen with a first inner diameter greater than the outer diameter of the elongated tubular member, the shrink tube adapted to shrink upon heating whereby the shrink tube after shrinking includes a second inner diameter of a desired width.

12. The kit of claim 11 wherein the die is formed near to a distal end of the shaft.

13. The kit of claim 11 wherein the die is formed at a proximal end of the shaft.

14. The kit of claim 11 wherein the die of the shaping tool includes a first curve, a second curve, and a third curve, the shape including a maximum outer diameter greater than a diameter of the shaft

15. The kit of claim 11 wherein the die forms a uniform convex curvature in an outer surface of the shaft.

16. The kit of claim 11 wherein the shaping tool includes a stop.

17. The kit of claim 11 wherein the shaft of shaping tool includes a second smaller outer diameter between the die and a stop.

18. The kit of claim 11 wherein the tubular member is made from a material selected from the group consisting of polyethylene, polyurethane, polypropylene, silicone rubber, and polyether block amide.

19. A shaping tool for molding an elongated tubular member into a desired shape comprising:
a cylindrical member with a first portion of an outside diameter approximately equal to an inner diameter of a lumen of the elongated tubular member and a second portion of an outside diameter greater than the inner diameter of the lumen of the elongated tubular member; and
a die of a selected contour disposed at a position on the first portion of the cylindrical member proximal to the second portion of the cylindrical member.

20. The shaping tool of claim 19 wherein the shaping tool is made of one or more of polytetrafluorethylene, acetal, or stainless steel.

21. The shaping tool of claim 19 wherein the die is circular.

22. The shaping tool of claim 19 wherein the die is pear shaped.

23. The shaping tool of claim 19 wherein the diameter of the second portion is greater than an outside diameter of the elongated tubular member.
